(19) United States
(12) Patent Application Publication
(10) Pub. No.: US 2010/0217086 A1
(43) Pub. Date: Aug. 26, 2010

(54) INFLATABLE SURGICAL RETRACTOR

(75) Inventors: Vivek R. Deshmukh, Vienna, VA (US); Neil R. Crawford, Tempe, AZ (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(73) Assignee: Catholic Heathcare West (CHW), Phoenix, AZ (US)

(21) Appl. No.: 12/393,568

(22) Filed: Feb. 26, 2009

(51) Int. Cl. A61B 1/32

(52) U.S. Cl. 600/205

(57) ABSTRACT

An expandable surgical retractor for minimally invasive surgical applications is disclosed. The expandable retractor, preferably in the form of an inflatable balloon retractor, is inserted in a surgical corridor and expanded to the desired size and shape. The retractor of the present invention is amenable to many shapes including cylindrical, conical with the base at the depth of the corridor, hourglass, and crescent and are dictated by the surgeon's needs. Cooling of the retractor allows the retractor to maintain the expanded characteristic. The expanded relatively rigid retractor provides an ideal corridor for surgical applications. In a preferred embodiment, a second retractor, of the invention, can be placed at a greater depth through the first placed retractor; the “telescoping” effect rapidly provides greater exposure with minimal manipulation. Following surgery, such retractor(s) can be removed in a manner that minimizes bleeding and tissue damage. One such method is reheating to soften and restore the retractor to its unexpanded size. Or, laterally placed perforations allow for fracturing of the retractor by bovie cautery, facilitating removal by “unzipping” the retractor.

### CROSS-REFERENCE TO RELATED APPLICATIONS

This invention claims priority to U.S. Provisional Patent Application No. 60/824,234, titled “INFLATABLE SURGICAL RETRACTOR”, filed Aug. 31, 2006. The content of this] application is incorporated by reference into this application as if fully set forth herein.

### FIELD OF THE INVENTION

The present invention concerns surgical devices used to maintain a surgical corridor. More particularly the present invention concerns a retractor created by materials having thermoplastic properties that permit the creation of a stable opening through which surgery can be performed.

### BACKGROUND OF THE INVENTION

It is desirable when surgery is required, or in any medical procedures, to be as minimally invasive as possible. The well being of the patient and speed of recovery are often dependent on the degree to which a procedure is quickly and accurately accomplished, with as little damage to the body and with as little blood loss as possible. For this reason laparoscopic and other minimally invasive surgical procedures have gained considerable favor among health care professionals.

Providing surgical procedures with minimally invasive openings from the skin, or other surface, to the point of surgical interest will tend to aid in the rapid recovery of the patient. The use of modern surgical techniques, including laparoscopy, fluoroscopy, MRI, CT and other methods of viewing and working within the operating theater, have made a significant difference in the quality and speed of patient recovery.

However, techniques for accomplishing such surgery have often been hampered by the need to provide a stable opening from an outer surface, such as the skin or the muscle through to the area of surgical interest, without causing damage to tissue there between. Presently it is necessary to form an incision and then by using mechanical retractors, pull back and hold an opening open throughout the surgical procedure. Such use of mechanical retractors tends to cause damage to skin surfaces and increases the time of recovery and pain that the patient feels. Further, the size of the incision needed to create the appropriately sized opening through which surgery will proceed can increase the amount of bleeding and oozing in the wound, cause tears in skin and muscle and provide a site for post-operative infections.

It would be desirable to conduct a surgical procedure using modern minimally invasive methods while providing a stable opening that can be made with minimal damage to the surrounding tissue.

### SUMMARY OF THE INVENTION

In accordance with the present invention, a surgical retractor for creating and maintaining an enlarged surgical corridor is provided. The retractor comprises a tube of thermally responsive material having a length sufficient to span from a skin surface through to a point of surgical interest. The thermally responsive material being pliable and expansive when heated above body temperature and becoming rigid when cooled to body temperature such that the tube can be heated to pliability and inserted into a narrow opening in the skin and through to the point of surgical interest, expanded by expanding means while pliable and in situ and then cooled or allowed to cool so as to maintain the enlarged opening thereby forming a surgical corridor.

In preferred embodiments, the tube can be generally cylindrical or conical in shape, as will be described in greater detail below. Further, the thermally responsive material in preferred embodiments becomes pliable at between 20° F. and 60° F. above body temperature and in some embodiments the thermally responsive material is a thermoplastic material. To aid in viewing the device in situ the thermally responsive material can be made of a radio-opaque material such that it is viewable in fluoroscopy. In such embodiments, the thermally responsive material can be of a type that is transparent in its pliable shape and radio-opaque in its rigid state; further the distal tip can be made radio-opaque to allow fluoroscopic verification of its placement in situ.

The invention further includes a method of expanding and fixing the circumference of a surgical corridor which comprises the steps of providing one or more tube of thermally responsive material, as described above, having either solely or in unison a length sufficient to span from an entry point of a patient to a point of surgical interest. Then heating the one or more tubes so as to make them pliable and expandable and inserting the heated, one or more tubes between the entry point, such as at a skin surface, and the point of surgical interest. Thereafter expanding the tube to form, solely or in unison, a surgical corridor between the entry point and point of surgical interest through which surgery may proceed.

The invention further includes means to quickly and easily remove the retractor following the end of the surgical procedure and a method for treating the retractor surfaces, prior to insertion, to help stave infection and provide a quicker recovery with faster healing. As well as a device that can be used to help perform all of the heating, expansion and cooling functions to the retractor; in the form of an expansion device having means to provide heated solution, expansion capabilities, and cooling solution sequentially to create the necessary pliability, enlarge the retractor and then cool the retractor to fix it into position during the surgical procedure.

A more detailed explanation of the invention is provided in the following description and claims and is illustrated in the accompanying drawings.

### BRIEF DESCRIPTION OF THE DRAWINGS

FIG. 1 is perspective view of a retractor made in accordance with the teachings of the present invention.

FIGS. 1A-FIG. 1C are perspective views of another retractor made in accordance with the teachings of the present invention.

FIG. 1D is a perspective view, partially broken away to show length, of another retractor made in accordance with the teachings of the present invention.

FIG. 1E is a cross-sectional view of a portion of the retractor of FIG. 1D, taken along the line 1E-1E thereof.

FIG. 1F is a perspective view, partially broken away to show length, of a number of retractors made in accordance with the teachings of the present invention shown fitted together to show a method of increasing the length of a surgical corridor.

FIG. 2 is schematic view of a retractor of FIG. 1 associated with an expansion device to make a balloon retractor construct.

FIG. 2A is a schematic view of a retractor of FIG. 1 associated with a light source and a light sensor.

FIG. 3 is a schematic representation of a surgical site, with a surgical corridor formed therein.

FIG. 4 is a schematic representation of a retractor and expansion device inserted together within the surgical corridor in an unexpanded state.

FIG. 5 is a schematic representation of a balloon retractor construct inserted within the surgical corridor in an expanded state.

FIG. 6 is a schematic representation of an expanded retractor within the surgical corridor.

FIG. 7 is a schematic representation of a device for heating and expanding the retractor of the present invention.

FIG. 8 is a plan view of a device of FIG. 7.

FIG. 9 is a perspective view of another device for expanding a retractor of the present invention.

### DETAILED DESCRIPTION OF THE ILLUSTRATIVE EMBODIMENT

While the present invention is susceptible of embodiment in various forms, there is shown in the drawings a number of presently preferred embodiments that are discussed in greater detail hereafter. It should be understood that the present disclosure is to be considered as an exemplification of the present invention, and is not intended to limit the invention to the specific embodiments illustrated. It should be further understood that the title of this section of this application (“Detailed Description of an Illustrative Embodiment”) relates to a requirement of the United States Patent Office, and should not be found to limit the subject matter disclosed herein.

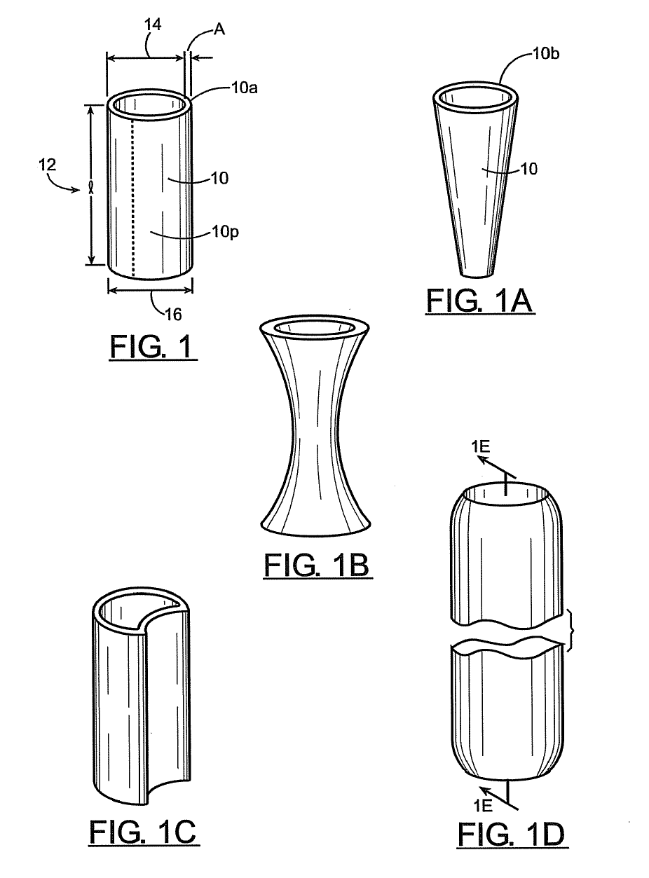

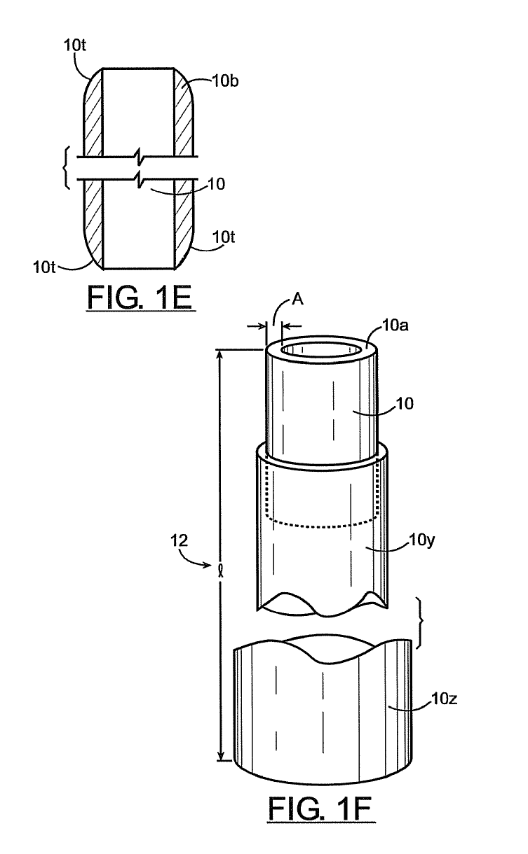

Referring to the drawings, specifically FIG. 1, a tube 10 of thermally responsive material, such as thermoplastics, is provided, having a length 12, an initial internal diameter 14 and an initial external diameter 16. The initial inner and outer diameters, 14, 16, of tube 10 define a tube thickness “A”. Tube 10, while shown as generally cylindrical, in FIG. 1, can be made such that it is generally conical, as shown in FIG. 1A, or in many other shapes, including for example the shapes shown in FIGS. 1B and 1C as will be understood by persons having ordinary skill in the art.

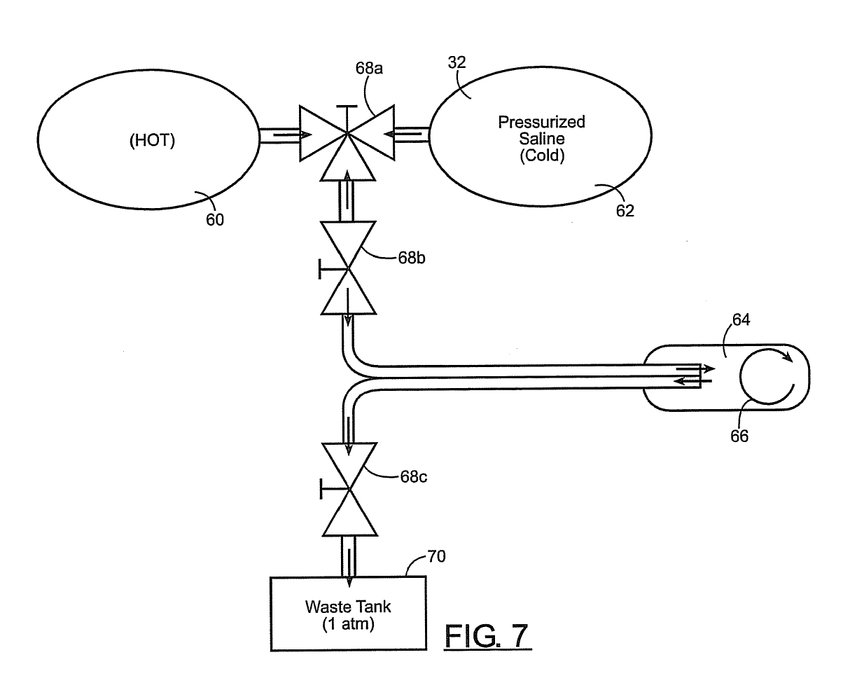

In one embodiment of the present invention, tube 10 is comprised of a rigid material that becomes pliable upon heating. Tube 10 can be heated, as will be discussed in greater detail below, until it reaches a desired state of pliability, such that the inner diameter 14 can be expanded to create an enlarged surgical corridor 18a as shown in FIG. 6. The pliable tube 10 may then be introduced into a surgical corridor 18 and a low compliance balloon 20 (of a balloon catheter 20a), of the type used in other surgical procedures, such as balloon angioplasty, is fitted within tube 10. It will be understood by persons having ordinary skill in the art that a combined tube 10 and balloon catheter 20a, or balloon retractor construct 25, can also be prepared together as a single surgical device.

Once tube 10 has been introduced into the surgical corridor 18 the balloon 20 can be filled with liquid or gas, though a syringe or other pressure forming means, causing expansion of the pliable tube 10. The walls 10a can be expanded to a desired size before allowing tube 10 to cool. Once cooled, tube 10 will be relatively rigid and the desired size will be maintained. The balloon 20 can then be deflated and removed thereby creating a surgical corridor 18a, FIG. 6, of a size and shape appropriate for the particular application. It will be understood by persons having skill in the art that the size of the surgical corridor 18a will be determined by the judgment of the surgeon and that a larger or smaller corridor can be made as needed without departing from the novel scope of the present invention.

It will be understood, by persons having ordinary skill in the art, that preferred material for tube 10 will be relatively pliable at a determined temperature range and relatively rigid at a lower temperature. In one embodiment, tube 10 can be comprised of thermoplastic material. Thermoplastics that become pliable around 20° F. to 60° F. higher than body temperature (98° F. or 37° C.) and rigid upon cooling are preferred. Protoplast™, a product of ProtoPlast, Inc. of Ontario Canada, is an exemplary thermoplastic commercially available and with a transition temperature of 140° F.

In another embodiment of the invention, tube 10 can be comprised of a radio-opaque material, thereby allowing a surgeon to easily see, on fluoroscopy, whether tube 10 has been installed appropriately before or after inflation. For example, a generally translucent thermoplastic, of a type well know to persons having ordinary skill in the art, that is transparent when soft, and opaque when relatively rigid, is a preferred material for use in the present invention. In another embodiment, the distal tip 25a of the balloon retractor construct 25 may be comprised of a radio-opaque material allowing fluoroscopic verification of the appropriate surgical site level.

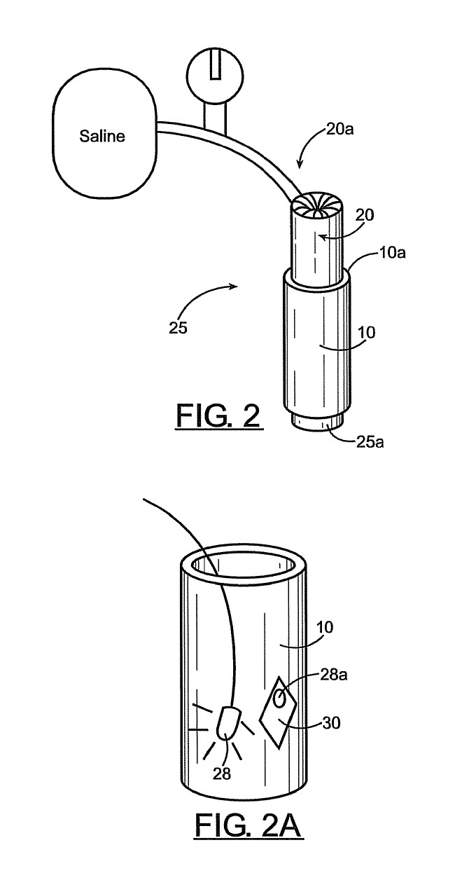

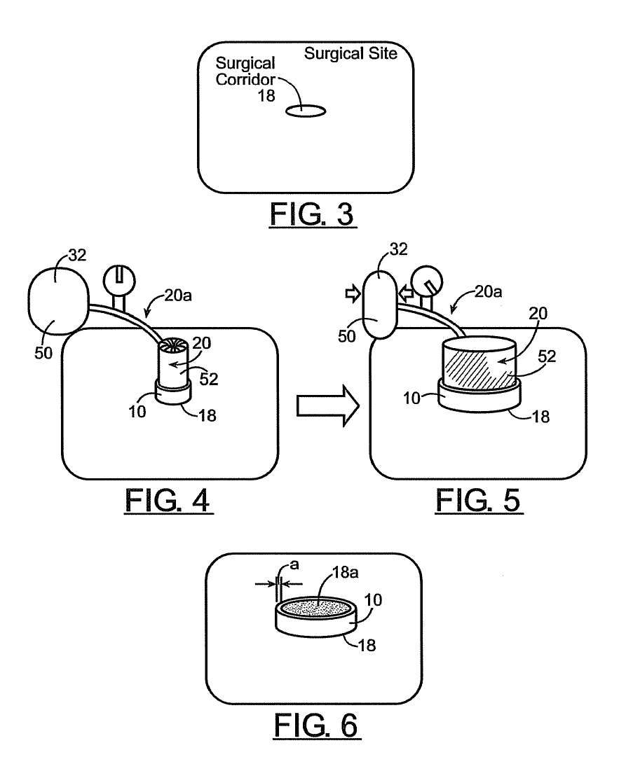

The translucence of tube 10 can also assist the surgeons and attendants with an automation of the device of the present invention. In another embodiment, shown in FIG. 2a, of the present invention, a small light 28, such as an LED, fiber optic cable or laser, and an accompanying optical sensor 30, fitted in tube 10, can be used to sense the wavelength of light reflected from tube 10. The light sensor 30 can indicate, for example by an indicator light 28a, when the walls 10a are opaque or translucent.

The shape of tube 10 can vary depending on the application and includes cylindrical, conical, as shown in FIGS. 1 and 1a, respectively, with the base 10b of the cone placed so that it is at the depth of the surgical corridor that is the site of surgical interest. Other shapes, including hourglass or bowtie (FIG. 1B), and crescentic (FIG. 1C) are possible as well. The shape of tube 10 maybe dictated by the shape of the balloon 20 in an expanded state or the differential composition of tube 10 which allows for preferential expansion in a particular location or direction. In one embodiment of the invention, tube 10 is cylindrical in shape. In some applications, however, it may be desirable to utilize a retractor with, for example, an hourglass or bowtie shape instead of a cylinder. An hourglass shape would be less vulnerable to displacement during surgery. A conical shape with the base at the depth of the field allows the surgeon to improve surgical exposure at the point of pathology while maintaining a small skin incision. The area and angles of exposure are greatly increased without enlargement of the skin incision. Simply manipulating the operating table or the surgical microscope facilitates the operative exposure. A crescent shape allows the surgeon to specifically enlarge the area of exposure in a given direction and minimizes unnecessary exposure. In some cases, a non-cylindrical retractor can be used to improve the strength of tube 10 in the hardened state. The composition of tube 10 can be selected to influence the final shape and strength of the expanded retractor. In one embodiment of the invention, the thickness of tube 10 is non-uniform such that an applied force at the interior of tube 10 will result in differential expansion of the walls to achieve a desired final shape.

In another embodiment of the invention, tubes 10 are comprised of a mixture of materials with different elasticity properties. The material mixture may be uniform or vary along a linear axis. In one embodiment of the invention, a ring of material with relatively low elasticity can be placed at the midpoint of a cylindrical, unexpended retractor. A cylindrical balloon 20 used to expand tube 10 will achieve greater expansion of the walls where the resistance to the applied force is less. In an expanded condition, the center portion of tube 10 will have a smaller diameter than the proximal and distal portions. Consistent with the scope of the invention, the shape of tube 10 in an unexpanded and in an expanded state can vary to accommodate any application and is not limited by specific embodiments described. Furthermore, it will be apparent to one skilled in the art that the composition and physical characteristics of tube 10 may be varied in any way to achieve a desired shape.

As described, heating of tube 10 enhances pliability. In one embodiment of the invention, tube 10 is heated to increase pliability prior to insertion in the surgical corridor. Tube 10 can be heated in a sterile water bath prior to insertion. Alliteratively, tube 10 can be heated by introducing a liquid of an appropriate temperature into a balloon fitted within tube 10. It will be readily apparent to one skilled in the art that in appropriate circumstance tube 10 can be inserted into the surgical corridor in a relatively rigid state and pliability obtained by infusing liquid in the balloon 20 after insertion. Consistent with the scope of the invention, the heating of tube 10 can be accomplished in any manner consistent with sterile surgical practices and is not limited to the provided descriptions.

The length 12 of tube 10 can also be varied depending on the application (see FIG. 1F). Because deformation of tube 10 will tend to occur radially, the overall length of tube 10 can be made relatively constant compared with the radial deformation. The length of the cylinder can, therefore, be selected by the surgeon to best suit the depth of retraction needed. Tube 10 length may be determined by the surgeon with the use of a depth gauge placed during the initial dissection. This depth gauge may also serve the dual purpose of allowing for fluoroscopic localization. In one embodiment of the invention, the surgeon can “telescope” retractor devices one partially within another. In one application, a first superficial corridor can be made and a retractor placed to the bottom of the initial dissection and expanded. Then, working within the proximal corridor, a more distal corridor is prepared that is continuous with the proximal corridor.

A second retractor 10y can then be placed deeper than the full extent of the first retractor and expanded such that the proximal few millimeters of the new retractor is in contact with the distal few millimeters of the first retractor. This sequence may continue to whatever final depth is needed. Telescoping allows the surgeon to carry out the procedure in steps conforming to the existing anatomic planes. For example, the first retractor 10 can be placed to the level of the fascia, retracting skin and subcutaneous fat. A fascial incision is then made, and a second and/or third retractor 10y-10z is telescoped to retract the fascia, muscle, and deep tissues. This sequence leads to exposure of the pathology of interest. Telescoping is particularly useful for the obese patient where a single size does not provide adequate depth of exposure. In one application, the interface between consecutive retractors can be made smooth by allowing an inflatable balloon to expand and deform the malleable retractor walls at the junctions to create smooth joints between telescoping retractors.

Consistent with the scope of the invention, other characteristics of tube 10 may be modified for specific applications. For example, in one embodiment of the invention, shown in FIG. 1E, the walls of tube 10 may be beveled 10t at the leading and trailing edges. A beveled leading edge may be desirable in some applications where the unexpanded device must be forced through a narrow initial corridor and a blunt leading edge would make this initial insertion difficult. A beveled trailing edge of tube 10 may also be desirable in some applications to prevent formation of a blunt trailing edge after expansion that may interfere with introduction of surgical instruments or additional retractors.

Tube 10 can also be fitted with a balloon 20. In one embodiment, a balloon 20 can be used to expand the pliable retractor walls 10a after insertion in the surgical corridor 18. In some applications, bulging of the balloon at the proximal and distal ends of tube 10 may reduce the efficiency of the balloon in producing the desired radial or outward expansion of the walls. The elasticity of the balloon can be chosen to reduce or prevent undesirable bulging. For example, the balloon can be comprised of cloth, such as is used for blood pressure cuffs, or an inelastic plastic film commonly used in some catheter applications. When choosing balloon material, the thermal gradient across the balloon also can be considered. For example, if the transition temperature of tube 10 is 140° F. but the thermal gradient across the balloon membrane is 20° F., then the liquid within the balloon needs to be at 160° F. if the balloon is being used to cause transitioning for expansion or removal of tube 10 (as described below).

The shape of the balloon will influence the final shape of tube 10. One skilled in the art will see that a variety of balloon shapes may be used depending on the circumstances and applications. In one embodiment, a cylindrical balloon 20 is used to create cylindrical retractor walls and a corresponding cylindrical surgical corridor 18. In other applications, conical or crescent shaped balloons may be used, depending on the application. In still other embodiments, the balloon may be shaped to allow for tapering at the proximal and/or distal ends of tube 10. An outward tapered end, or more of an hourglass/bowtie shape instead of a cylinder, may be a desirable retraction shape that can have a beneficial role in maintaining a stable retractor position superior to a straight cylinder. One skilled in the art will see that the applications and balloon shapes are not limited by the disclosures.

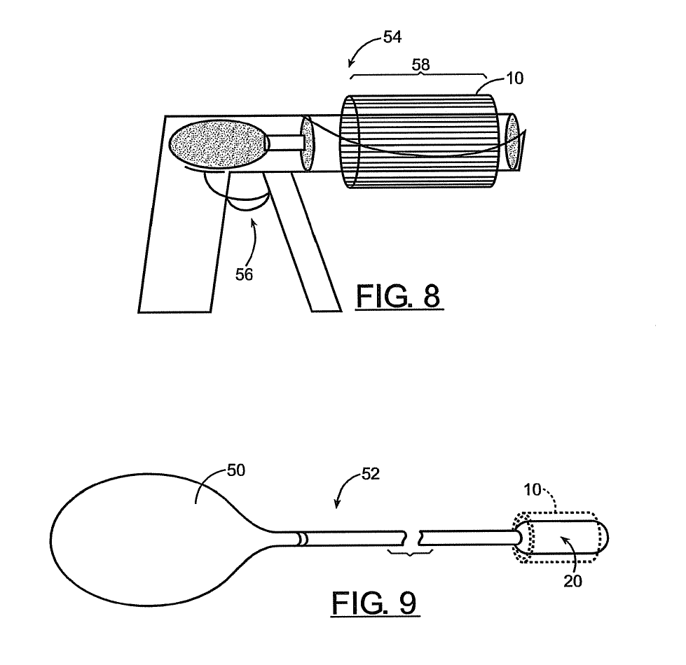

As previously described, a fluid 32 can be used to fill and expand the balloon. In one embodiment of the invention, a saline solution is used to protect the patient in the case of accidental rupture of the balloon material. The saline solution in this embodiment is introduced using technology similar to that currently used for inflating low compliance balloon catheters with pressurized saline. Alternatively, saline solution may be delivered via an infusion pump syringe controlled with on/off forward/reverse switches (not shown). In yet another embodiment of the invention, shown in FIG. 9, a simple squeeze bag 50 that the operator manually compresses may be used to eject saline solution or some other fluid from a bag and into the insufflator 52. In another embodiment, shown in FIG. 8, tube 10-balloon construct can be loaded onto the shaft of a hand-held device 54 shaped like a “gun.” The trigger 56 is coupled with balloon inflation. The surgeon places the construct 58 within the wound and deploys tube 10 by pulling the “trigger.” A new retractor 10 can be loaded for a second deployment. Introduction of a fluid into the balloon can be accomplished in a variety of ways, as will be readily seen by one skilled in the art.

A more complex temperature-controlled embodiment of the invention can also be utilized, as shown in FIG. 7. In one embodiment, a hot liquid 60 and a cold liquid 62 alternately are used to accelerate the solidification process and facilitate device removal. Such an embodiment can utilize a balloon 64 that is not only fillable with pressurized liquid, for example a saline solution, but also circulates 66 the liquid through the balloon. Circulating the liquid continuously also is believed to confer additional advantages because the body tissues around tube 10 tend to create a sizeable heat sink and the heat transfer from tube 10 to the liquid within the balloon may not be adequate to counter such a large heat sink. Such a device would have valve means 68 with which to choose whether cold or hot fluid was being circulated within the balloon 64. Further a waste tank 70 would be provided so that fluid, previously circulated within balloon 64, could be removed therefrom, for example so as to deflate balloon 64 once retractor 10 is expanded to its desired size. Engineering of a balloon with circulating hot or cold saline requires the balloon to have an inlet valve 68b from pressurized liquid and an outlet valve 68c. One or both of the valves have variable dimension. With variable rates of inflow and/or outflow, three conditions are achieved: (1) with more flow through the inlet valve than the outlet valve, pressure in the balloon would be created causing the balloon to inflate, (2) with equal inlet and outlet valve flow rates, the balloon would maintain its volume but would have circulating liquid within, (3) with greater flow through the outlet valve 68c than the inlet 68b, the balloon 64 deflates. A switch 68a for the inlet liquid supply enables the user to select hot or ice-cold saline, 60, 62 respectively. For inflating the balloon to its largest dimension, hot saline circulates in the balloon. For accelerating the transition of the thermoplastic, it may be desirable to switch to cold saline. For removing the retractor at the end of the procedure, hot saline may again be used to reinflate the balloon and soften the thermoplastic.

Following surgery, tube 10 can be removed in a variety of ways. In some applications, tube 10 can be removed by simply pulling tube 10 out of the surgical canal 18. In other applications, pulling tube 10 out will cause undesirable tissue damage at the surgical site. To avoid tissue damage, in one embodiment of the invention, tube 10 can be made of a material having a transition temperature low enough to be tolerated by the tissues. In these applications, the surgical corridor may be filled with heated saline to cause softening of the malleable retractor walls and allow easy removal. In embodiments of the invention using small diameter unexpanded thermoplastic retractors, the thermoplastic material returning to its original shape upon re-heating will aid removal. Upon introduction of heated saline, tube 10 would return to a small diameter for easy removal. In other embodiments of the invention, a heating wire or a tool with a heated tip may be used to create seams in tube 10 from the inside. Following creation of seams, tube 10 may be broken out of the surgical corridor in pieces. Tube 10 can carry additional perforations laterally 10p to facilitate this “unzipping” maneuver. Such an application would be particularly useful, for example, where the transition temperature of tube 10 material is so high that introduction of heated saline solution into the corridor would cause undesired tissue damage.

In some applications, bleeding may occur upon removal of tube 10. In one embodiment of the invention, shown in FIG. 5, bleeding is mitigated by coating the tube 10 and/or balloon 20 (or other insufflating means) with a hemostatic agent 52 such as, for example, gel foam prior to insertion. For example, the tube 10 and balloon 20 could be coated by rolling them in a tray of the hemostatic agent. In some applications, it may also be necessary to utilize lubricants, sheaths, or other coatings to prevent adhesion of the tube and insufflator to surrounding tissues. Such adhesion may occur when using a hemostatic agent in some applications.

It will be readily recognized by one skilled in the art that the invention may be optimized and is well suited for usage with a surgical robot. A surgical robot can be utilized to hold and position a rigid straight or curved extension with the balloon and retractor on its tip, then move the balloon into exact position based on medical images. It can also be used, for example, to precisely adjust the rotational position of the balloon if an asymmetrical balloon (for example, half-cylinder shape) is needed for a particular refraction. Both position and orientation may be more accurately controlled by a surgical robot than manually in certain applications.

Although an illustrative embodiment of the invention has been shown and described, it is to be understood that various modifications and substitutions may be made by those skilled in the art without departing from the novel spirit and scope of the invention.

What is claimed is:

1. A surgical retractor for creating and maintaining an enlarged surgical corridor, comprising:
a tube of thermally responsive material having a length sufficient to span from a skin surface through to a point of surgical interest;
the thermally responsive material being pliable and expansive when heated above body temperature and becoming rigid when cooled to body temperature such that the tube can be heated to pliability and inserted into a narrow opening in the skin and through to the point of surgical interest, expanded by expanding means while pliable and in situ and then cooled or allowed to cool so as to maintain the enlarged opening thereby forming a surgical corridor.

2. The surgical retractor of claim 1, wherein the shape of the expanded tube is dictated in part by varying the width of the walls of the retractor and/or the composition of the material in a portion of the wall in a specified region of the wall.

3. The surgical retractor of claim 1, wherein the expanding means is a balloon.

4. The surgical retractor of claim 3, wherein the shape of the expanded tube is dictated in part by using a balloon having a specified shape

5. The surgical retractor of claim 1, wherein the tube is generally cylindrical in shape.

6. The surgical retractor of claim 1, wherein the tube is generally conical in shape.

7. The surgical retractor of claim 1, wherein the tube is generally hour-glass shaped.

8. The surgical retractor of claim 1, wherein the tube is generally crescentic in shape.

9. The surgical retractor of claim 1, wherein the thermally responsive material becomes pliable at between 20° F. and 60° F. above body temperature.

10. The surgical retractor of claim 1, wherein the thermally responsive material is a thermoplastic material.

11. The surgical retractor of claim 1, wherein the thermally responsive material is a radio-opaque material such that when in situ the surgical retractor is viewable in fluoroscopy.

12. The surgical retractor of claim 11, wherein the thermally responsive material is transparent in its pliable shape and radio-opaque in its rigid state.

13. The surgical retractor of claim 1, wherein the distal tip is radio-opaque to allow fluoroscopic verification of its placement in situ.

14. The surgical retractor of claim 1, wherein an illumination means and a light detection means are included in the retractor to provide an indication of placement in situ.

15. A surgical retractor for creating and maintaining an enlarged surgical corridor, comprising:
a cylindrical tube of thermally responsive material having a length sufficient to span from a skin surface through to a point of surgical interest;
the thermally responsive material being pliable and expansive when heated, to about between 20° F. and 60° F., above body temperature and becoming rigid when cooled to body temperature such that the tube can be heated to pliability and inserted into a narrow opening in the skin and through to the point of surgical interest, expanded by expanding means while pliable and in situ and then cooled or allowed to cool so as to maintain the enlarged opening thereby forming a surgical corridor.

16. The surgical retractor of claim 15, wherein the thermally responsive material is a thermoplastic material.

17. The surgical retractor of claim 15, wherein the thermally responsive material is a radio-opaque material such that when in situ the surgical retractor is viewable in fluoroscopy.

18. The surgical retractor of claim 15, wherein the thermally responsive material is transparent in its pliable shape and radio-opaque in its rigid state.

19. The surgical retractor of claim 15, wherein the distal tip is radio-opaque to allow fluoroscopic verification of its placement in situ.

20. A method of expanding and fixing a surgical corridor comprising the steps of:
providing one or more tube of thermally responsive material having either solely or in unison a length sufficient to span from an entry point of a patient to a point of surgical interest; warming the one or more tube so as to be pliable and expandable;
inserting the one or more tube between the entry point and the point of surgical interest; and,
causing the one or more tube to expand to form solely or in unison a surgical corridor between the entry point and point of surgical interest.

21. A method of expanding and fixing a surgical corridor comprising the steps of: providing one or more tube of thermally responsive material having either solely or in unison a length sufficient to span from an entry point of a patient to a point of surgical interest;
inserting the one or more unexpanded tube between the entry point and the point of surgical interest;
warming the one or more tube in situ so as to be pliable and expandable; and,
causing the one or more tube to expand to form solely or in unison a surgical corridor between the entry point and point of surgical interest.

22. The method of expanding and fixing a surgical corridor of claim 18 including the step of providing a triggerized means to heat, expand and inflate the device in situ.

23. The method of expanding and fixing a surgical corridor of claim 18 including the step of using the means to expand and inflate to deflate and reduce the one or more tube so that the one or more tube can be removed.
